SEQUENCE LISTING (10 sequences)

SEQ ID NO: 1 - DNA, 891 bp, Arabidopsis thaliana
atggatttctggccggagtttatggcgaccagctggggaagagagttcgtcgccggtggtttcggtggcgtcgccggcatcatctccggctaccccttggacaccctcagaattcgccagcaacagagctcgaaatctggatctgccttctccattctccggcggatgctcgccattgagggtccctcctctctctacagaggcatggctgcgcctttggcctccgtcacttttcagaatgctatggtattccagatatacgccatattctctcgctcttttgattcctctgttcctctggtagagcctccttcctacagaggcgttgctcttggtggtgttgccaccggtgctgtacagagcctcttgctcacccctgtcgagctcatcaagattcgtctccagcttcagcagactaagtctggtcccatcaccttggccaagagcatccttaggagacagggccttcaggggctttacagaggcctcaccatcaccgtgctccgagatgctcccgctcatggcctctacttctggacctatgagtacgtcagagaaaggcttcaccccggctgcagaaagaccggacaagaaaacctcaggaccatgctcgtggctggtggccttgctggagtggccagctgggtcgcttgttatcctcttgatgtcgtcaagaccagactccaacaaggtcatggggcttacgagggcattgccgattgttttcgcaagagcgtcaaacaggaaggctatacggttctctggcgtggcctcgggactgcagttgccagggcctttgtggtcaacggtgctatctttgctgcttatgaggtagccttgcggtgtctattcaatcaatcgccatccccagatattgtcacaggagattga

SEQ ID NO: 2 - protein, 296 aa, Arabidopsis thaliana
MDFWPEFMATSWGREFVAGGFGGVAGIISGYPLDTLRIRQQQSSKSGSAFSILRRMLAIEGPSSLYRGMAAPLASVTFQNAMVFQIYAIFSRSFDSSVPLVEPPSYRGVALGGVATGAVQSLLLTPVELIKIRLQLQQTKSGPITLAKSILRRQGLQGLYRGLTITVLRDAPAHGLYFWTYEYVRERLHPGCRKTGQENLRTMLVAGGLAGVASWVACYPLDVVKTRLQQGHGAYEGIADCFRKSVKQEGYTVLWRGLGTAVARAFVVNGAIFAAYEVALRCLFNQSPSPDIVTGD

SEQ ID NO: 3 - DNA, 930 bp, Arabidopsis thaliana
atggcgacgagaacggaatcgaagaagcagattccgccgtacatgaaagcagtctcaggctcactaggcggagtggtcgaggcttgttgtctccaaccaatcgacgtaatcaaaacgcgtctccagctagatcgcgtcggcgcttacaaaggaatcgctcactgtggttcgaaggtggttcgcaccgaaggagttcgtgctctctggaaaggcttaacaccgttcgctactcatctcacgcttaagtacacgcttcggatgggatccaacgccatgtttcaaaccgcctttaaggattccgagaccggaaaggtcagcaatcgtggccgttttctttccggattcggtgccggtgttcttgaagctctcgccattgttacaccctttgaggtggtgaaaattagacttcagcagcagaaaggattgagtcctgagcttttcaagtacaaaggaccaatacattgtgctagaaccatcgtgagagaagaaagcatacttggtttatggtcaggtgcagcaccgacggttatgcgaaacggaaccaaccaagctgtaatgttcacagcgaaaaacgcgtttgacatactcttgtggaacaaacacgaaggtgacggtaaaatcttgcagccatggcagtcaatgatctcagggtttttagctggaaccgcaggtccgttctgcacaggaccgtttgatgtggtgaaaacgaggttgatggctcagagcagagacagtgaaggtgggattagatataaagggatggttcatgccattagaacgatttatgcagaggaaggattagtggccttatggagagggttattgccgaggctaatgaggattcctccaggacaagccattatgtgggctgttgctgatcaagtcactggtctttatgagatgagatatcttcggaatgctcctttatag

SEQ ID NO: 4 - protein, 309 aa, Arabidopsis thaliana
MATRTESKKQIPPYMKAVSGSLGGVVEACCLQPIDVIKTRLQLDRVGAYKGIAHCGSKVVRTEGVRALWKGLTPFATHLTLKYTLRMGSNAMFQTAFKDSETGKVSNRGRFLSGFGAGVLEALAIVTPFEVVKIRLQQQKGLSPELFKYKGPIHCARTIVREESILGLWSGAAPTVMRNGTNQAVMFTAKNAFDILLWNKHEGDGKILQPWQSMISGFLAGTAGPFCTGPFDVVKTRLMAQSRDSEGGIRYKGMVHAIRTIYAEEGLVALWRGLLPRLMRIPPGQAIMWAVADQVTGLYEMRYLRNAPL

SEQ ID NO: 5 - DNA, 33 bp, Arabidopsis thaliana
agcctcgagatggatttctggccggagtttatg

SEQ ID NO: 6 - DNA, 30 bp, Arabidopsis thaliana
ttcggatcctcaatctcctgtgacaatatc

SEQ ID NO: 7 - DNA, 29 bp, Arabidopsis thaliana
acgctcgagatggcgacgagaacggaatc

SEQ ID NO: 8 - DNA, 32 bp, Arabidopsis thaliana
acggcggccgcctataaaggagcattccgaag

SEQ ID NO: 9 - DNA, 33 bp, Arabidopsis thaliana
ttcggatccatggatttctggccggagtttatg

SEQ ID NO: 10 - DNA, 31 bp, Arabidopsis thaliana
aatcccgggatctcctgtgacaatatctggg

(19) United States
(12) Patent Application Publication
(10) Pub. No.: US 2004/0137438 A1
(43) Pub. Date: Jul. 15, 2004

(54) Nucleic acids, by means of which plants with altered metabolite content can be produced

(76) Inventors: Elisabetta Catoni, Boblingen (DE); Wolf-Bernd Frommer, San Francisco, CA (US); Rebecca Schwab, Tubingen (DE); Karin Shumacher, Tubingen-Haggelloch (DE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/333,243

(22) PCT Filed: Jul. 20, 2001
(86) PCT No.: PCT/EP01/08379

(30) Foreign Application Priority Data
Jul. 27, 2000 (DE) 100 36 671.6

(51) Int. Cl. C12Q 1/68; C07H 21/04; C07K 14/705

(52) U.S. Cl. 435/6; 435/69.1; 435/320.1; 435/325; 530/350; 536/23.2; 435/317.1

(57) ABSTRACT

The invention relates to a nucleic acid that encodes a plant transporter of the inner mitochondrial membrane, in particular a member of the mitochondrial carrier family (MCF), and uses of this nucleic acid. The invention furthermore relates to a fragment of the nucleic acid, a construct containing the nucleic acid or a fragment thereof, and a host cell that contains the nucleic acid, the fragment, or the construct. The present invention furthermore relates to a method for producing a transgenic plant by using the nucleic acid, a fragment thereof, or a construct containing the nucleic acid or a fragment thereof, as well as a method for modulating the transport properties of the inner mitochondrial membrane of a plant, a plant part, a plant cell and/or seeds.

### FIELD OF THE INVENTION

The present invention relates to a nucleic acid that encodes a plant or animal transporter of the inner mitochondrial membrane, in particular a member of the mitochondrial carrier family (MCF), as well as its use for modifying the mitochondrial transport, in particular in plants. The present invention furthermore relates to a fragment of the nucleic acid, a construct containing the nucleic acid or a fragment thereof, as well as a host cell, in particular a plant cell that contains the nucleic acid or a fragment thereof. The present invention furthermore relates to a transgenic plant, methods for producing a transgenic plant, as well as methods for modifying the transport properties of the inner mitochondrial membrane of a plant, in particular of a plant part.

### BACKGROUND OF THE INVENTION

Transporters play a special role in the function of an organism. They decide the taking-up or release of a substance into or from a cell or organism, and in this way control the transport and distribution of the substances between the cells. On the level of the intracellular substance distribution, the transporters of the organelle membranes have an important regulatory function, since individual steps of a metabolic pathway frequently take place in different compartments, and the intermediates must be transported between these compartments.

Mitochondria, also called the power plants of the cell, are especially important compartments. In addition to the respiration and photorespiration processes, a number of anabolic and catabolic steps of different metabolic pathways also take place in the mitochondria. The citrate cycle, which, as a central “hub”, controls the flow of the basic carbon structures for a number of metabolites, is also located in the mitochondria. It is therefore obvious that numerous metabolites, for example, pyruvate, amino acids, carboxylic acids, and fatty acids, must be transported into and out of the mitochondria. In contrast to the outer mitochondrial membrane that is freely passable for many substances, the inner mitochondrial membrane has transport systems with a high specificity. One group of these transport systems includes carriers that work according to the principle of exchange (antiport). Examples of this include the adenyl nucleotide carrier that exchanges exogenous ADP for internal ATP, dicarboxylic acid carrier that exchanges malate for 2-oxoglutarate, and tricarboxylic acid carrier that exchanges citrate for malate. Other substances, such as glutamate and aspartate are transported without exchange (uniport), while the transport mechanism is not yet known for several other metabolites. The transport systems of the inner mitochondrial membrane enable the metabolite exchange and are therefore essential for integrating the cytosolic and mitochondrial compartment. The majority of transporters of the inner mitochondrial membrane, identified so far primarily at a molecular level in yeast and in animal systems, belong to the mitochondrial carrier family (MCF) that is characterized by its preserved protein structure and the presence of a defined signature sequence (mitochondrial energy transfer sequence) (Palmieri, FEBS Letters, 346 (1994), 48-54). Based on phylogenetic analyses, the MCF representatives are divided into a total of 17 sub-families, whereby substrates so far are known for only 5 sub-families (Moualij et al., Yeast 13 (1997), 573-581).

A large family of nuclear-coded genes appears to encode representatives of this protein class also in higher plants. However, so far only the isolation and characterization of the ADP/ATP translocator in a number of plant types and of the malate/2-oxoglutarate translocator from Panicum has been accomplished. It is interesting that the plastid adenylate translocator encoded by the brittle-1 gene of maize is also an MCF member. An analysis of this protein family in higher plants thus could be useful not only for researching the mitochondrial transport, but also for the plastid transport (Sullivan et al., Plant Cell, 3 (1991), 1337-48; Shannon et al., Plant Physiol., 117 (1998), 1235-52).

By analyzing the yeast mutants arg11 (Crabeel et al., J. Biol. Chem., 271 (1996), 25011-25018) and acr1 (Palmieri et al., FEBS Lett., (1997), 114-118), two MCF members were identified that function as transporters for arginine/ornithine or, respectively, for succinate/fumarate.

Biosynthesis and breakdown of the amino acid arginine take place with involvement of mitochondrial and cytosolic enzymes so that both arginine itself as well as its precursor ornithine must pass through the inner mitochondrial membrane. The arg11 mutant of the yeast was isolated as an arginine-auxotrophic mutant, i.e., it is not able to grow on medium without arginine. Cloning of the ARG11 gene and the biochemical characterization of its gene product showed that this is an MCF member that is able to transport both ornithine and arginine. In higher plants, the amino acid arginine represents a preferred storage form for nitrogen because of its high nitrogen content. For example, in the germ layers of the soybean, 60% of the nitrogen present in the form of free amino acids and 18% of the nitrogen present in seed proteins exists as arginine. The identification of intracellular transporters, in particular of the mitochondrial arginine transporter, is a prerequisite for being able to perform specific modifications of the protein content and/or the protein composition of crop plants.

The citrate cycle taking place in the mitochondria is in the center of the metabolism of all organisms. The intermediate removed from the citrate cycle as biosynthetic precursors must be replenished by so-called anaplerotic reactions. The glyoxylate cycle taking place in the cytosol or in the cytosols is the most important anaplerotic reaction both in yeast as well as in plants, since the succinate resulting from this reaction is used to replenish the citrate cycle. For this purpose, the succinate first must be transported from the cytosol into the mitochondria, however. In yeast, this transport is performed by the ACR1 protein, which is also an MCF member. In the absence of ACR1, yeast cells are unable to grow on acetate or ethanol as a sole carbon source since the succinate resulting from their catabolism does not reach the mitochondria. In plants, the transport of succinate into the mitochondria is a critical step in the mobilization of the carbon stored in the form of fatty acids. The acetyl-CoA from the β-oxidation is converted in the glyoxisomes into succinate, which then can be introduced into the citrate cycle. The efficiency and speed with which the stored energy can be mobilized is of central importance for the germination of fat-storing plant seeds. This means that in plants, manipulations of the transport of succinate into the mitochondria can be used for a specific modification of the germination behavior.

### BRIEF SUMMARY OF THE INVENTION

The present invention has the underlying technical objective of providing methods and means for identifying and/or isolating plant or animal transporters of the inner mitochondrial membrane, in particular plant MCF members, as well as further methods and means based on these methods and means in order to be able to specifically modify plants with respect to a modified, mitochondrial transport of metabolic metabolites and potentially also a modified plastid transport of metabolic products. The specific modification of the mitochondrial transport in a preferred embodiment relates in particular to the arginine transport in crop plants in order to specifically modify their protein content and/or their protein composition, and to the transport of succinate in order to specifically modify the germination behavior of plants.

According to the invention, the technical objective was realized in that genes encoding transporters of the inner mitochondrial membrane, in particular plant MCF members, and the proteins encoded by them were identified and characterized by way of complementation of the yeast mutants arg11 and arc1. It was hereby shown for the first time that a plant protein of the inner mitochondrial membrane is also correctly localized in yeast, i.e., reaches its destination. According to the invention, a preferably isolated and completely purified nucleic acid is provided, selected from the group comprising:

a) a nucleic acid obtainable by complementation of MCF-deficient host cells with nucleic acid sequences from a plant or animal gene bank and by selection of MCF-positive host cells, or a fragment thereof;

b) a nucleic acid with a nucleotide sequence shown in SEQ ID No. 1 or in SEQ ID No. 3, or a fragment thereof;

c) a nucleic acid with a sequence encoding a protein with a sequence shown in SEQ ID No. 2 or in SEQ ID No. 4, or a fragment thereof;

d) a nucleic acid that is complementary to a nucleic acid according to a) to c), or a fragment thereof;

e) a nucleic acid obtainable by substitution, addition, inversion, and/or deletion of one or more bases of a nucleic acid according to a) to d); and

f) a nucleic acid that because of the degeneration of the genetic code hybridizes with a nucleic acid according to a) to e) or a fragment thereof.

A nucleic acid may be a DNA sequence, for example, a cDNA or genomic DNA sequence, or an RNA sequence, for example, an mRNA sequence.

In connection with the present invention, the terms “MCF” member and “MCF protein” stand for a protein with MCF activity, i.e., a protein involved in the transport of metabolites through the inner mitochondrial membrane, potentially also through the inner plastid membrane, and which has the typical characteristics of this protein family. The typical characteristics are on the one hand the primary structure that consists of a triple repeat of a domain comprising about 100 amino acids with two each membrane spans, as well as the existence of up to three repeats of the so-called “mitochondrial energy transfers” signature (P-X-(D/E)-X-(LIVAT)-(RK)-X-(LRH)-(LIVMFY). The terms “MCF member” and “MCF protein” in connection with the present invention include in particular transporter molecules for arginine/ornithine and transporter molecules for succinate/fumarate. To demonstrate the transporter activity, for example, the method described by Palmieri et al. (FEBS Letters, 417 (1997), 447) may be employed.

In connection with the present invention, the terms “MCF-deficient cells” or “MCF-deficient host cells” stand for cells that because of one or more genetic defects have negatively modified, mitochondrial transporter properties that result in a negatively selectable phenotype. In MCF-deficient plant cells, the plastid transport potentially also may be modified negatively.

In connection with the present invention, “MCF-positive cells or host cells” therefore mean cells that contain either naturally a nucleic acid encoding an MCF member, or cells that because of a complementation of MCF-negative cells contain a nucleic acid which at least partially compensates the defect(s) causing the negatively modified mitochondrial transport, and which therefore demonstrate a positively selectable phenotype.

The term “complementation” used in the present invention stands for a compensation of a genetic functional defect reflected in the phenotype of an organism, while preserving the mutation(s) causing the defect. A complementation in connection with the invention, for example, exists when a genetic defect in an MCF gene (for example, in the ARG11 gene of Saccharomyces cerevisiae) is compensated by the presence of a similar, intact gene (for example, the ATARG11 gene from Arabidopsis thaliana), whereby the intact gene assumes the function of the defective MCF gene.

### BRIEF DESCRIPTION OF THE DRAWINGS

The following figures and examples will explain the invention:

FIG. 1a shows a hydrophobicity analysis according to Kyte-Doolittle for the ARG11 and at ARG11 proteins,

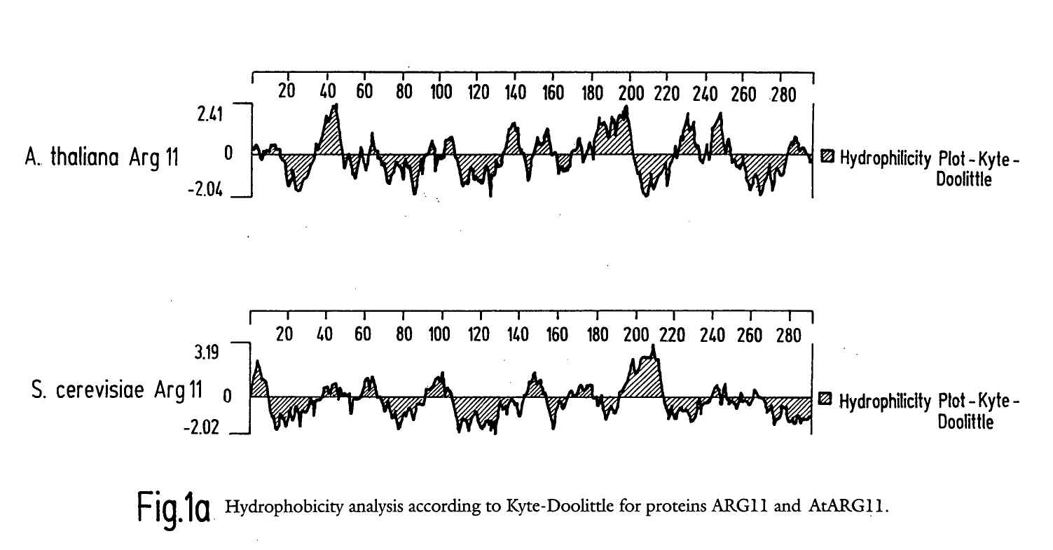

FIG. 1b shows a schematic of the structure of an MCF member using the example of AtARG11,

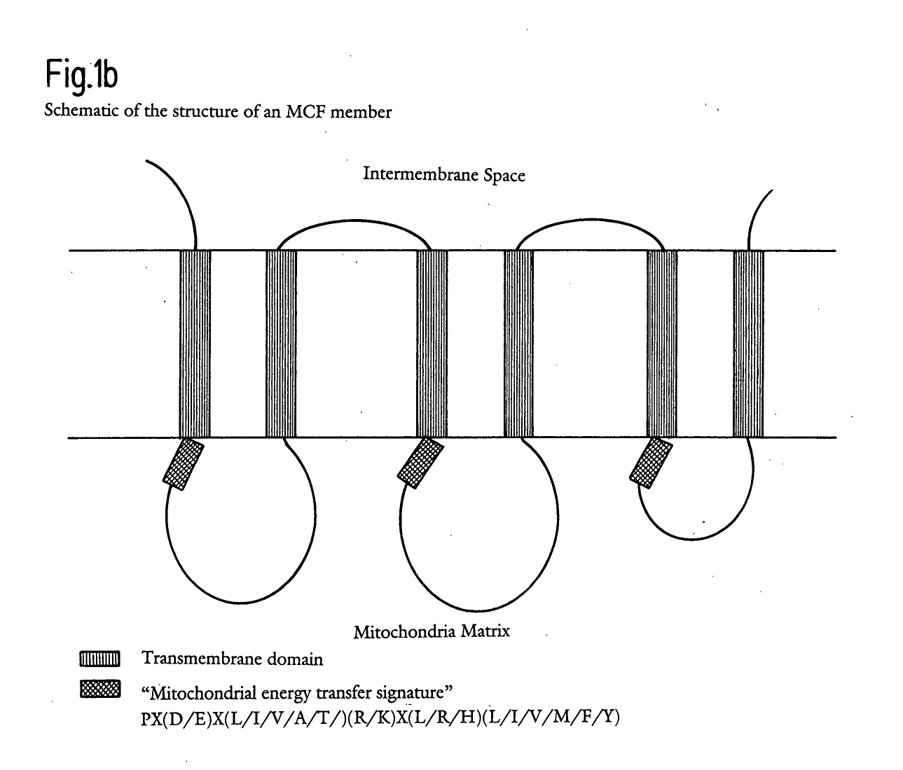

FIG. 2 shows the complementation of the yeast mutant arg ARG11 [sic] by expression of the ATARG11 gene under control of the PMA promoter

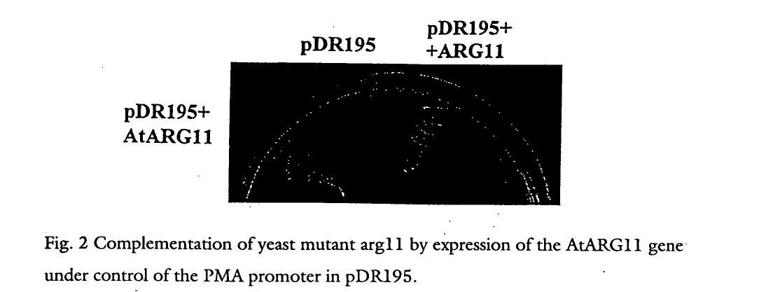

FIG. 3 shows the complementation of the yeast mutant acr1 by expression of the ATACR1 gene under control of the PMA promoter.

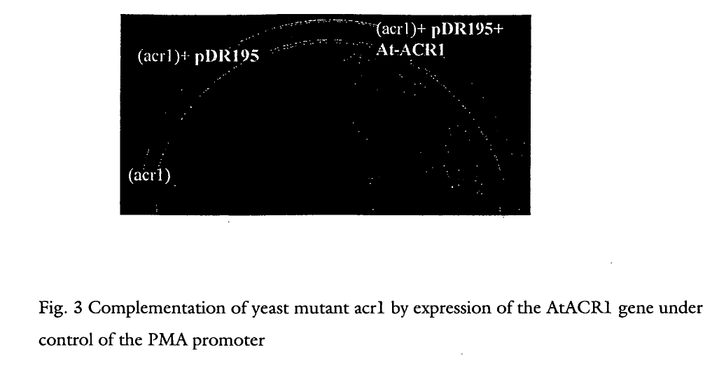

### DETAILED DESCRIPTION OF THE INVENTION

According to the invention, MCF-deficient cells or host cells are used in preferred embodiments for the isolation and identification of nuclei c acids that encode an MCF member, in particular a transporter molecule for arginine/ornithine or a transporter molecule for succinate/fumarate. Preferred MCF-deficient host cells are eukaryotic cells, for example, plant or animal cells, preferably yeast cells. The identification according to the invention of an MCF member takes place by complementation of specific mutations in MCF-deficient host cells, in particular of specific mutants of the yeast Sacchoramyces cerevisiae. In this process, nucleic acid sequences of a plant or animal gene bank, for example, a cDNA bank or genomic bank, are transformed into MCF-deficient host cells, after which a selection for MCF-positive host cells takes place.

The isolation of a gene that encodes a specific transporter molecule requires suitable yeast mutants, which, due to a defect in this transporter molecule, are unable to transport a certain substance into the mitochondria. Delforge et al. (Eur. J. Biochem., 57 (1975), 231), for example, describes the mutant arg11 (MG409 strain) that can only grow in media containing the amino acid arginine. In addition, in order to perform the complementation with plant or animal genes, the URA3 gene was destroyed, whereby a uracil auxotrophy was created (MG409ura3Γ, 1c1636d). This means that this mutant also needs uracil for growth.

In order to provide-a nucleic acid according to the invention, a suitable yeast mutant, for example, the arg11/ura3 mutant, is transformed with expression plasmids, suitable for use in yeast, which contain the cDNA fragments from a plant or animal cDNA gene bank. Sequences that encode the plant or animal mitochondrial transporter molecules can be identified by selecting transformants, which are able, due to the expression of these plant or animal cDNA sequences, to grow on medium without arginine. According to the invention, nucleic acids that encode proteins with sequence homology into already known mitochondrial transporter molecules can be cloned into suitable yeast expression plasmids and be tested for their ability to complement specific mutations. The invention therefore also relates to the previously described methods for identifying and/or isolating nucleic acids that encode a protein with the activity of an MCF protein, whereby MCF-deficient host cells are transformed with nucleic acid sequences of a plant or animal gene bank, MCF-positive host cells are selected, and the MCF-encoding nucleic acids present in the positive host cells are isolated and/or identified using standard methods.

Using the arg11/ura3 yeast mutant and suitable expression plasmids that contain the promoter of the proton ATPase PMA1 of yeast, cDNA clones that cause a complementation of the yeast mutation were isolated from Arabidopsis thaliana. An analysis using sequencing and restriction methods as well as other biochemical-molecular-biological methods found that these Arabidopsis thaliana cDNA fragments isolated according to the invention encode plant MCF members. The nucleic acid sequences of these cDNA clones are shown in SEQ ID No. 1 and SEQ ID No. 3, and the corresponding amino acid sequences of these cDNA clones are described in SEQ ID No. 2 and SEQ ID No. 4.

The invention also relates to modified nucleic acids that can be obtained, for example, by substitution, addition, inversion and/or deletion of one or more bases of a nucleic acid according to the invention, in particular within the encoded sequence of a nucleic acid, i.e., it also includes nucleic acids that can be called mutants, derivatives, or functional equivalents of a nucleic acid according to the invention. Such manipulations of the sequences are performed, for example, in order to specifically modify the amino acid sequence encoded by a nucleic acid, for example, by changing the specificity of a mitochondrial transporter. Nucleic acids that encode modified mitochondrial transporters also can be used for the transformation of agriculturally used plants in order to create transgenic plants. Specific sequence modifications also may be performed with the objective of providing suitable restriction sites or removing not required nucleic acid sequences or restriction sites. The nucleic acids according to the invention are hereby inserted into plasmids and are subjected to a mutagenesis or sequence modification by recombination using standard microbiological/molecular-biological methods. In order to create insertions, deletions, or substitutions, such as transitions and transversions, suitable methods include, for example, methods for in vitro mutagenesis, primer repair methods, as well as restriction and/or ligation methods (cf Sambrook et al., 1989, Molecular Cloning: A Laboratory Manual, 2nd Edition (1989), Cold Spring Harbor Laboratory Press, NY, USA). Sequence modifications also can be achieved by addition of natural or synthetic nucleic acid sequences. Examples of synthetic nucleic acid sequences are adapters or linkers, which also are added to these fragments for linking nucleic acid fragments to these fragments.

The present invention furthermore relates to nucleic acids that hybridize with one of the previously described nucleic acids under a) to e). The expression “nucleic acid that hybridizes with a nucleic acid under a) to e)” used in connection with the present invention stands for a nucleic acid that hybridizes with a nucleic acid under a) to e) under moderately stringent conditions. For example, the hybridization may take place with a radioactive gene probe in a hybridization solution (25% formamide; 5×SSPE; 0.1% SDS; 5× Denhardt solution; 50 μg/ml herring sperm DNA; with respect to the composition of individual components, refer to Sambrook et al., Molecular Cloning: A Laboratory Manual, 2nd edition (1989), Cold Spring Harbor Laboratory Press, NY, USA) for 20 hours at 37° C. After this, unspecifically bound probe is removed by washing the filters several times in 2×SSC/0.1% SDS at 42° C. The filters preferably are washed with 0.5×SSC/0.1% SDS, in particular with 0.1×SSC/0.1% SDS at 42° C.

The present invention also relates to nucleic acids that encode a polypeptide or protein with MCF activity, whose sequence has at least 40%, preferably at least 60%, and in particular at least 80% homology with a polypeptide or protein encoded by a nucleic acid with a sequence shown in SEQ ID No. 1 or SEQ ID No. 3.

In connection with the invention, the expression “at least 40%”, preferably at least 60%, in particular at least 80% homology” relates to a sequence match on the amino acid sequence level that can be determined using known methods, for example, computer-based sequence comparisons (Basic local alignment search tool, S. F. Altschul et al., J. Mol. Biol. 215 (1990), 403-410).

The expression “homology” known to the expert designates the degree of relationship between two or more polypeptide molecules that is determined by the match between the sequences, whereby match may mean both an identical match as well as a conservative amino acid exchange. The percentage of “homology” results from the percentage of matching ranges in two or more sequences, with consideration of gaps or other peculiarities of the sequences.

The expression “conservative amino acid exchange” stands for the exchange of an amino acid remnant for another amino acid remnant, whereby this exchange does not result in a change of polarity or charge at the position of the exchanged amino acid. One example for a conservative amino acid exchange is the exchange of a nonpolar amino acid remnant for another nonpolar amino acid remnant. In connection with the invention, conservative amino acid exchanges include, for example:

G=A=S, I=V=L=M, D=E, N=Q, K=R, Y=F, S=T

G=A=I=V=L=M=Y=F=W=P=S=T.

The homology between interrelated polypeptide molecules can be determined by using known methods. As a rule, special computer programs with algorithms accounting for the special requirements are used. Preferred methods for determining the homology first produce the greatest match between the studied sequences. Computer programs for determining the homology between two sequences include, but are not limited, the GCG program suite, including the GAP (Devereux, J., et al., Nucleic Acids Research, 12 (12) (1984), 387; Genetics Computer Group University of Wisconsin, Madison (Wis.)); BLASTP, BLASTN, and FASTA (S. Altschul et al., J. Mol. Biol. 215 (1990), 403-410). The BLASTX program is available from the National Centre for Biotechnology Information (NCBI) and from other sources (Altschul S., et al., BLAST Manual, NCB NLM NIH Bethesda, Md. 20894; Altschul, S. et al., J. Mol. Biol 215 (1990), 403-410). The known Smith Waterman algorithm also can be used to determine the homology.

Preferred parameters for the sequence comparison include, for example:

| Algorithm: | Needleman and Wunsch, J. Mol. Biol 48 (1970), |
|---|---|
| | 443-453; |
| Comparison matrix: | BLOSUM 62 by Henikoff and Henikoff, Proc. |
| | Natl. Acad. Sci. USA, 89 (1992), 10915-10919; |
| Gap penalty: | 12 |
| Gap length penalty: | 4 |
| Similarity threshold: | 0 |

The GAP program also is suitable for using the previously described parameters. The previously described parameters are default parameters for amino acid sequence comparisons.

In addition, further algorithms, gap opening penalties, gap extension penalties, and comparison matrices, including those described in the program manual of the Wisconsin suite, version 9 (September 1997). The selection of programs depends both on the comparison to be performed as well as on whether the comparison is performed between sequence pairs, whereby GAP or Best Fit are preferred, or between a sequence and an extensive sequence database, whereby FASTA or BLAST are preferred.

The present invention also relates to a preferably isolated and completely purified protein available by expressing a nucleic acid according to the invention or a fragment thereof in a host cell. The protein preferably has the same transport properties as the protein encoded by a nucleic acid with a sequence shown in SEQ ID No. 1 or SEQ ID No. 3. In order to establish the activity of such a protein, for example, uptake experiments, as described below in the exemplary embodiment, may be performed.

The present invention also relates to isolated and completely purified monoclonal or polyclonal antibodies or their fragments, which react with a protein according to the invention.

The invention furthermore relates to a construct containing a nucleic acid according to the invention and/or a fragment thereof under control of expression regulatory elements. In connection with the present invention, the term “construct,” which also may be called a vector here, stands for the combination of a nucleic acid according to the invention or a fragment thereof, with at least one additional nucleic acid element, for example, a regulatory element. Examples of such regulatory elements are constitutive or inducible promoters, such as the E. coli promoter araBAD (Carra and Schlief, EMBO J., 12 (1993), 35-44) for expression in bacteria, the yeast promoter PMA1 (Rentsch et al., FEBS Lett., 370 (1995), 264-268) for expression in fungal systems, and the viral CaMV35S promoter (Pietrzak et al., Nucl. Acids Res., 14 (1986), 5857-5868) for expression in plants. The nucleic acid or the fragment furthermore may be provided with a transcription termination signal. Such elements have already been described (cf, for example, Gielen et al., EMBO J., 8 (1984), 23-29). The transcription start and termination sites can be native (homologous) or foreign (heterologous) to the host organism. The sequence of the transcription start and termination sites can be of synthetic or natural origin or may include a mixture of synthetic and natural components. In an especially preferred embodiment of the invention, the construct is a plasmid.

The nucleic acid or the fragment may exist in the construct, in particular in a plasmid, both in a antisense as well as in a sense orientation to the regulatory element(s). If the nucleic acid or the fragment is located, for example, in sense orientation to the regulator element, for example, a promoter, it may inhibit or reduce the activity of the endogenous transporter of the inner mitochondrial membrane through co-suppression effects, in particular after transformation and integration in higher numbers of copies into the genome. If the nucleic acid or fragment is located in the construct in antisense orientation to the regulator element, the construct may be inserted, for example, into the genome of a plant host cell and may result in a suppression of the formation of the inherent plant mitochondrial transporter molecules after its transcription.

A preferred embodiment of the present invention therefore comprises a construct that includes a nucleic acid according to the invention or a fragment thereof in antisense orientation to a promoter, whereby the expression of a mitochondrial transporter molecule or, as the case may be, the expression of a plastid transporter molecule, is inhibited in a host cell containing the construct. The nucleic acid fragment hereby includes at least 10 nucleotides, preferably at least 50 nucleotides, in particular at least 200 nucleotides. The construct that contains a nucleic acid according to the invention or a fragment thereof can be inserted into a host cell and can be transcribed there into a non-translatable RNA (antisense RNA) that is able, by binding to an endogenous gene for a mitochondrial transporter or to the mRNA transcribed from it, to inhibit the expression of this endogenous gene.

Another preferred embodiment of the invention relates to a construct that contains a nucleic acid according to the invention or a nucleic acid fragment according to the invention in sense orientation to a regulator element, for example, a promoter, which is followed by another, identical or different nucleic acid according to the invention or another identical or different nucleic acid fragment according to the invention in antisense orientation to it. This arrangement enables the formation of a double-strand RNA able to induce the breakdown of the endogenous RNA (Chuang and Meyerowitz, Proc. Natl. Acad. Sci. USA, 97 (2000), 4985-90; Sijen and Kooter, Bioessays, 22, (2000), 520-531).

In another preferred embodiment of the invention, the plasmid contains a replication signal for E. coli and/or yeast, and a marker gene that permits a positive selection of the host cells transformed with the plasmid. If the plasmid is inserted into a plant host cell, additional sequences, which are known to one skilled in the art, may be required, depending on the insertion method. If the plasmid, for example, is a derivative of the Ti or Ri plasmid, the nucleic acid to be inserted or the fragment thereof must be flanked by T-DNA sequences that enable the integration of the nucleic acid or of the fragment thereof into the plant genome. The use of T-DNA for the transformation of plant cells has been extensively studied and has been described, for example, in EP 120 516; Hoekema, The Binary Plant Vector System, chapter V (1985), Offset-drukkerij Kanters B. V. Ablasserdam; in Fraley et al., Crit. Rev. Plant. Sci., 4 (1985), 1-46, and in An et al., EMBO J., 4 (1985), 277-287. Once the inserted nucleic acid or fragment thereof is integrated into the genome, it is usually stable there and is also preserved in the progeny of the originally transformed cell.

The sequence integrated in the genome also may include a selection marker that, for example, provides the transformed plant cells with resistance to a biocide or an antibiotic, such as kanamycin, G418, bleomycin, hygromycin, or phosphinotricin. The used marker permits the selection of transformed cells versus cells without the transformed DNA.

The invention thus also provides a host cell containing a nucleic acid according to the invention, in particular a nucleic acid with a sequence shown in SEQ ID No. 1 or a nucleic acid with a sequence shown in SEQ ID No. 3, or a fragment thereof, or a construct containing a nucleic acid according to the invention or a fragment thereof. The host cell according to the invention may be a bacterium or a yeast, insect, mammal, or plant cell.

The present invention furthermore relates to a transgenic plant containing in at least one of its cells a nucleic acid according to the invention that encodes a protein with MCF activity, or a fragment thereof, or a construct according to the invention. The transgenic plants may be plants of different species, genera, families, orders, and classes, i.e., both monocotyle as well as dicotyle plants, as well as algae, mosses, ferns, or gymnosperms. Transgenic plants also may include calli, plant cell cultures, as well as parts, organs, tissues, and harvest or propagation materials of these plants. In the present invention, the transgenic plants are in particular tobacco, potato, tomato, sugar beet, soybean, coffee, pea, bean, cotton, rice, or maize plants.

In connection with the present invention, the expression “in at least one of its cells” means that a transgenic plant contains at least one cell, but preferably a plurality of cells, that contain one or more nucleic acids according to the invention or a fragment thereof or a construct according to the invention which have been integrated in a stable manner. The nucleic acid or a fragment thereof or a construct according to the invention may be integrated both in the nucleus of the cell or in the mitochondrial or plastid genome. The nucleic acid, fragment, or construct preferably is integrated in a location of the cell that does not correspond to its natural position, or is integrated with a number of copies and/or in an orientation that does not correspond to the naturally occurring number of copies and/or orientation.

The present invention also relates to a method for producing a transgenic, preferably fertile plant that comprises the following steps:

Insertion of a nucleic acid according to the invention, in particular of a nucleic acid with a sequence shown in SEQ ID No. 1 or a nucleic acid with a sequence shown in SEQ ID No. 3, or a fragment thereof, or a construct containing a nucleic acid according to the invention or a fragment thereof, into a plant cell;

Regeneration of a preferably fertile plant from the transformed plant cell, whereby at least one cell of this plant is transgenic, i.e., that contains a nucleic acid according to this invention integrated in a stable manner and preferably expresses it, i.e., transcribes the integrated nucleic acid to RNA. The resulting plant preferably has a modified activity of a transporter of the inner mitochondrial membrane in at least one of its mitochondria.

A number of methods are available for inserting a nucleic acid into a plant cell. Most of these methods require that the nucleic acid to be inserted is present in a construct, such as a vector. Vectors, for example, plasmids, cosmids, viri, bacteriophages, shuttle vectors, etc., are known. Vectors often comprise functional units for stabilizing the vector in a host cell and for enabling its replication in it. Vectors may also contain regulatory elements with which the nucleic acids are functionally bonded and which enable the expression of the nucleic acid.

In addition to the transformation using agrobacteria, for example, Agrobacterium tumefaciens, there are numerous other methods available. These methods include the fusion of protoplasts, microinjection of DNA, electroporation as well as biolistic methods and virus injection methods. In contrast to transformation using agrobacteria, injection and electroporation do not per se have any special requirements for the vector. Simple plasmids, such as, for example, pUC derivatives, can be used. However, if whole plants are to be regenerated from such transformed cells, the presence of a selectable marker gene is advantageous.

Then whole plants can be regenerated from the transformed plant cells in a suitable medium potentially containing antibiotics or biocides for selection. The resulting plants then can be tested for the presence of the inserted DNA. The transformed cells grow within the plants in the standard manner (cf. McCormick et al., Plant Cell Reports, 5 (1986), 81-84). These plants can be grown as usual and can be crossed with plants having the same transformed genetic traits or other genetic traits. The resulting hybrid individuals demonstrate the corresponding phenotypic properties.

The present invention furthermore relates to a method for modifying the properties of the mitochondrial and/or plastid transport of a plant, in particular for modifying the mitochondrial arginine/ornithine or succinate/fumarate transport in a plant cell, a plant tissue, a plant organ and/or a whole plant, whereby a nucleic acid according to the invention, in particular a nucleic acid with a sequence shown in SEQ ID No. 1 or a nucleic acid shown in SEQ ID No. 3, or a fragment thereof, are inserted into a plant cell and/or a plant, and then a whole plant is regenerated, whereby in at least one of its cells an expression of the transformed nucleic acid can take place so that a plant with modified metabolite content is obtained.

In order to modify the properties of the mitochondrial transport of a plant, both the specificity of the transport system—whereby the transport of new compounds is enabled—as well as the transport mechanism are modified. For example, the invention relates to modifications of the mitochondrial and/or plastid transport in a plant, within the context of which the affinity and/or substrate specificity of a transporter of the inner mitochondrial membrane, in particular of an MCF transporter molecule, is changed to the effect that a more efficient transport is achieved into the mitochondria or out of the mitochondria. This may be accomplished, for example, by bringing about an over-expression of the MCF transporter molecule, for example, by inserting several gene copies or by inserting constructs that contain the isolated nucleic acid sequence according to the invention under the control of a strong, constitutively or tissue- and/or time-specifically expressing promoter. The modifications of the mitochondrial transport also can be achieved by repression, suppression, and/or co-suppression of endogenous MCF genes, as a result of which, for example, targeted accumulations of specific substances inside or outside of the mitochondria or plastids occur. Such effects inhibiting the endogenously present MCF activity can be achieved according to the invention by transformation of plant cells with antisense constructs and/or by integration of several sense constructs in order to achieve a co-suppression effect and/or by using a knockout approach. Naturally, the activity of endogenously present genes of the transporters of the inner mitochondrial membrane also can be directly inhibited by using the nucleic acids according to the invention, for example, by integration into the endogenously present nucleic acid via homologous recombination.

The present invention furthermore relates to the use of the nucleic acids according to the invention for modifying the mitochondrial and/or plastid transport, in particular on the inner mitochondrial membrane of plants or on the inner plastid membrane of plants.

In a preferred embodiment, the invention makes available methods for using the previously mentioned nucleic acids, according to which methods, in particular, the amino acid and/or carboxylic acid transport on the inner mitochondrial membrane is modified.

In particular, the invention relates to methods for modifying the mitochondrial arginine and ornithine transport. The amino acid arginine represents the main storage form for nitrogen in the seeds and storage organs of many plants. During germination, the arginine integrated in storage proteins or present as free amino acid is split by the enzyme arginase first into ornithine and urea. The resulting urea is then broken down by urease into ammonium and CO₂. The released ammonium then is able to flow into the amino acid biosynthesis that is particularly important during germination. The two enzymes arginase and urease are localized in plant cells in the matrix of the mitochondria so that the arginine present in the cytosol first must be transported into the inner mitochondrial membrane.

An especially preferred embodiment of the invention thus provides the over-expression or ectopic expression of the plant mitochondrial arginine/ornithine transporter according to the invention under control of different promoters, for example, CaMV-35S, or seed-specific promoters, in order to increase the efficiency of the nitrogen mobilization through the arginine breakdown and in this way improve the germination behavior.

Ornithine is a precursor for the synthesis of alkaloids, such as nicotine, atropine, and cocaine. Thus, a modification of the alkaloid content can be achieved by changing the ornithine pool.

Another especially preferred embodiment of the invention thus provides the over-expression or ectopic expression and antisense inhibition of the plant arginine/ornithine transporter for modifying the alkaloid content.

The invention also relates to methods for modifying the mitochondrial succinate transport. In plants, the transport of succinate into the mitochondria is a critical step in the mobilization of the carbon stored in the form of fatty acids. The acetyl-CoA from the β-oxidation is converted in the glycosomes to succinate, which then can be introduced into the citrate cycle. The efficiency and speed with which the stored energy can be mobilized is of central importance particularly in the germination of fat-storing seeds.

A preferred embodiment of the present invention thus provides the over-expression or ectopic expression of the plant mitochondrial succinate transporter under control of different promoters, for example, CaMV-35S, or seed-specific promoters, in order to increase the efficiency of the energy gain from the β-oxidation of fatty acids and in this way improve the germination behavior of seeds.

The nucleic acids according to the invention, in particular a nucleic acid with a sequence shown in SEQ ID No. 1 or a nucleic acid with a sequence shown in SEQ ID No. 3 also may be used for isolating homologous sequences from bacteria, fungi, plants, animals and/or humans. In order to be able to search for homologous sequences, first gene banks must be created, for example, genomic banks or cDNA banks. With the help of a probe that contains parts of the previously mentioned nucleic acids, sequences then can be isolated from the gene banks. After identification and/or isolation of the corresponding genes, DNA and amino acid sequences can be determined, and the properties of the proteins encoded by these sequences can be analyzed.

The nucleic acids according to the invention also can be used to study the expression of a transporter according to the invention of the inner mitochondrial membrane, especially of an MCF member, in prokaryotic and/or eukaryotic cells. If the previously described nucleic acids, are inserted, for example, into prokaryotic cells, such as bacteria, a RNA sequence, translatable by bacteria, of a eukaryotic transporter of the inner mitochondrial membrane, in particular of a eukaryotic MCF member, is formed, which in spite of substantial differences in the membrane structures of prokaryotes and eukaryotes is translated into a functional eukaryotic MCF member with the substrate specificity of the latter. These bacteria cells therefore can be used for studies of the properties of a transporter molecule as well as of its substrates. According to the invention, the nucleic acids according to the invention can be used, under control of a regulatory element, in antisense direction for inhibiting the expression of an endogenous transporter of the inner mitochondrial membrane, in particular of an endogenous MCF member, in prokaryotic and/or eukaryotic cells. Another possible use of these nucleic acids is the production of transgenic crop plants.

The nucleic acids according to the invention or the MCF transporter molecules encoded by them furthermore can be used for studies of herbicides. Some of the transporter molecules essential for plants represent targets for herbicides, which may inhibit the function of the transporters. According to the invention, screening processes can be performed, especially in yeast, in order to search for inhibitors of the MCF transporters according to the invention. These inhibitors can be further tested in the yeast system and may be optimized by chemical modification. Then a test of these inhibitors on plants can be performed. The use of the MCF transporters according to the invention for studying herbicides is of particular interest to the extent that the mitochondrial carrier family is eukaryote-specific, so that an herbicide targeting an MCF member should not be harmful for soil bacteria. As a result of the comparatively high divergence between protein sequences of the MCFs from different species it is therefore possible to identify herbicides with a low toxicity for non-plant organisms.

Since the site of action of any herbicides is in the plastids or mitochondria, its transport there is a good starting point for developing new resistance mechanisms. The invention provides a targeted modification of the substrate specificity of plastid-localized MCF members according to the invention, making it possible to produce crop plants in which herbicides do not reach their site of action.

The sequence protocol includes:

SEQ ID No. 1 shows the DNA sequence (comprising 891 nucleotides) of the AtARG11 gene from Arabidopsis thaliana.

SEQ ID No. 2 shows the amino acid sequence (comprising 296 amino acids and derived from SEQ ID No. 1) of the mcf protein according to the invention.

SEQ ID No. 3 shows the cDNA sequence (comprising 930 nucleotides) of the ATACR1 gene from Arabidopsis thaliana.

SEQ ID No. 4 shows the amino acid sequence (comprising 309 amino acids and derived from SEQ ID No. 3) of another mcf protein according to the invention.

SEQ ID Nos. 5 to 10 show primers from Arabidopsis thaliana used for cloning.

### EXAMPLES

### Example 1

a) Cloning Methods:

The vector pDR195 (D. Rentsch et al., FEBS Lett., 370 (1995), 264-268) was used for cloning in E. coli and for transforming yeasts. For plant transformation, the gene construct was cloned into the binary vector CHF3, a derivative of pPZP212 (P. Hajdukiewicz et al., Plant Mol. Biol., 25 (1994),989-994).

The vector pSMGFP4 was used to localize the protein in plants.

b) Bacteria and Yeast Strains:

The E. coli strain DH5α was used to amplify and clone the various vectors.

1c1636d (arg11-1, ura3), a derivative of MG409 (ura3) (Delforge et al., Eur. J. Biochem., 57 (1975), 231), and G60 (acr1, ura3), a derivative of 23344c (ura3) (Grenson et al.) were used as a yeast strain.

c) Transformation of Agrobacterium tumefaciens:

The DNA transfer into the agrobacteria was performed by direct transformation according to the method by Hofgen and Willmitzer (Nucl. Acids Res., 16 (1988), 9877). The plasmid DNA of transformed agrobacteria was isolated according to the method by Birnboim and Doly (Nucl. Acids Res., 7 (1979), 1513-1523) and analyzed by gel electrophoresis after splitting with suitable restriction enzymes.

d) Plant Transformation:

The plant transformation can be performed with a gene transfer mediated by Agrobacterium tumefaciens (strain C58C1, pGV2260) (Deblaere et al., Nuc. Acids Res., 13 (1985), 4777-4788). The transformation of A. thaliana, for example, is performed by vacuum infiltration (modified according to Bechtold et al. (Comptes Rendus de l'Academie des Sciences Serie III, Sciences de la Vie, 316 (1993), 1194-1199). Pots (with a diameter of 10 cm) are filled with soil, after which a mosquito net is stretched across them. On this net, A. thaliana seeds are sown. These plants are used for the vacuum infiltration six to eights weeks after sowing. For the vacuum infiltration, 2×1 liters of cultures were grown from the corresponding agrobacterium strains in YEB medium that contained antibiotics (50 μg/ml kanamycin and 100 μg/ml rifampicin) at 28° C. With an OD₆₀₀ of 1.5, the cells were harvested at 3,000 g [maybe we should say “harvested by centrifugation at 3,000 g to make it clearer, even though centrifuging is not mentioned?] and were resuspended in 600 ml of infiltration medium (0.5×MS medium (Sigma), 5% saccharose, 44 μM benzylaminopurine). The bacteria suspension is filled into 250 ml glass canning jars and placed into a dehydrator. The A. thaliana plants are dipped “head first” into the bacteria suspension, and then a vacuum is applied for five minutes. The seeds of this plant are harvested after 3-4 weeks. For surface sterilization, the seeds are shaken for 10 minutes in 4% sodium hypochloride, 0.02% triton, centrifuged off at 1,500 g, washed four times with sterile water, and resuspended in 3 ml of 0.05% agarose (per 5,000 seeds). The seed-agarose solution is spread on MSS medium (1×MS, 1% saccharose, 0.8% agarose, 50 μ/ml kanamycin, pH 5.8) (13.5 cm diameter plates for 5,000 seeds). To reduce the moisture loss, the plates are closed with Parafilm®. The kanamycin-resistant plants are replanted in soil. Seeds of these plants are harvested and analyzed.

### Example 2

### Cloning of Arginine/Ornithine Transporter Gene from Arabidopsis thaliana

The AtARG11 gene was amplified by PCR from a cDNA gene bank of Arabidopsis thaliana with primers AtARG11-f (5′-agcctcgagatggatttctggccggagtttatg-3′, SEQ ID No. 5) and AtARG11-r (5′-ttcggatcctcaatctcctgtgacaatatc-3′, SEQ ID No. 6). The primer AtARG11-f contains an XhoI site, and the primer ATARG11-r contains a BamHI site. The PCR product was split with the restriction enzymes BamHI and XohI and ligated into the plasmid pDR 195, which also had been split with the restriction enzymes BamHI and XhoI. Then the plasmid was transformed in E. coli cells.

The insertions in the plasmids were sequenced. A DNA sequence with 891 nucleotides could be identified (SEQ ID No. 1). SEQ ID No. 2 shows the associated amino acid sequence of the arginine-ornithine transporter molecule.

Approximately 1 μg of the plasmid was transformed according to the method by Gietz (Gietz et al., Yeast 11 (1995), 355-360) into the yeast strain 1c1636d. Then yeast mutants that were able to grow on minimal medium without arginine were selected.

### Example 3

### Cloning of the Succinate Transporter Gene from Arabidopsis thaliana

The ATACR1 gene was isolated using the PCR method from a cDNA gene bank of Arabidopsis thaliana with the primer AtACR1-f (5′-acgctcgagatggcgacgagaacggaatc-3′, SEQ ID No. 7) that contains a site for the restriction enzyme XhoI, and the primer AtACR1-r (5′-acggcggccgcctataaaggagcattccgaag-3′, SEQ ID No. 8) that contains a NotI site.

The PCR product was split with the restriction enzymes XhoI and NotI and ligated into the plasmid pDR 195 that also had been split with the restriction enzymes XhoI and NotI. Then E. coli cells were transformed with the plasmid.

The insert of the plasmid was sequenced. A cDNA sequence comprising 930 nucleotides could be identified (SEQ ID No. 3). The amino acid sequence of the succinate transported encoded by this DNA sequence is shown in SEQ ID No. 4 and comprises 309 amino acids.

Approximately 1 μg of the plasmid was transformed according to the method by Gietz (Gietz et al., Yeast 11 (1995), 355-360) into the yeast strain G60. Then yeast transformants that were able to grow on minimal medium with ethanol as the sole carbon source were selected.

### Example 4

### Cloning of the AtARG1 and ATACR1 Genes into Vector CHF3

The ATACR1 gene and the AtARG11 gene were cloned into the vector CHF3 in order to achieve an over-expression in plants. The genes were cut from the corresponding pDR195 constructs with BamH1 and Xho1 and were ligated into the vector CHF3 (CHF3-ARG11) that was split with BamH1 and Sma1. The produced plasmids were transformed into Agrobacterium tumefaciens, and the resulting bacteria strains were used for the transformation of Arabidopsis.

For localization in plants, the AtARG11 gene was amplified by PCR from a cDNA gene bank of Arabidopsis thaliana with primers AtARG11-GFP-f (5′-ttcggatccatggatttctggccggagtttatg-3′, SEQ ID No. 9) and AtARG11-GFP-r (5′-aatcccgggatctcctgtgacaatatctggg-3′, SEQ ID No. 10). The primer AtARG11-GFP-f contains a BamHI site, and the primer AtARG11-GFP-r contains a Sma1 site. The PCR product that was split with BamHI and Sma1 was ligated into the vector pSMGFP4 that also had been split with BamHI and SmaI. The gene product was localized subcellularly by fluorescence microscopy.

1. Nucleic acid encoding a plant or animal transporter of the inner mitochondrial membrane, selected from the group consisting of:
a) a nucleic acid obtainable by complementation of MCF-deficient host cells with nucleic acid sequences from a plant or animal gene bank and by selection of MCF-positive host cells, or a fragment thereof;
b) a nucleic acid with a nucleotide sequence shown in SEQ ID No. 1 or a nucleic acid with a nucleotide sequence shown in SEQ ID No. 3, or a fragment thereof;
c) a nucleic acid with a sequence encoding a protein with a sequence shown in SEQ ID No. 2 or a protein with a sequence shown in SEQ ID No. 4, or a fragment thereof;
d) a nucleic acid that is complementary to a nucleic acid according to a) to c), or a fragment thereof;
e) a nucleic acid obtainable by substitution, addition, inversion, and/or deletion of one or more bases of a nucleic acid according to a) to d); and
f) a nucleic acid that because of the degeneration of the genetic code hybridizes with a nucleic acid according to a) to e) or a fragment thereof.

2. Nucleic acid according to claim 1, characterized in that it is a DNA or RNA.

3. Fragment of a nucleic acid according to claim 1 or 2, characterized in that it is able to inhibit the expression of a plant MCF member in a host cell in antisense orientation to a promoter.

4. Fragment according to claim 3, characterized in that it comprises at least 10 nucleotides, preferably at least 50 nucleotides, in particular at least 200 nucleotides.

5. Construct containing a nucleic acid according to claim 1 o 2 or a nucleic acid fragment according to claim 3 or 4 under control of expression regulatory elements.

6. Construct according to claim 5, characterized in that the nucleic acid or the fragment are in antisense orientation to the regulatory element.

7. Construct according to claim 5 or 6, characterized in that it is present in the form of a plasmid.

8. Host cell containing a nucleic acid according to claim 1 or 2, or a fragment thereof, or a construct according to one of claims 5 to 7.

9. Host cell according to claim 8, selected from bacteria, yeast cells, mammalian cells, and plant cells.

10. Transgenic plant containing a nucleic acid according to claim 1 or 2, or a fragment thereof, or a construct according to one of claims 5 to 7.

11. Transgenic plant, characterized in that the nucleic acid or the fragment or the construct are integrated at a position of the genome that does not correspond to its natural position.

12. Protein, obtainable by expression of a nucleic acid according to claim 1 or 2 in a host cell according to claim 8 or 9.

13. Antibody that reacts with a protein according to claim 12.

14. Method for producing a transgenic plant, comprising the following steps:
Insertion of a nucleic acid according to claim 1 or 2, or a fragment thereof, into a plant cell; and
Regeneration of a plant from the transformed plant cell.

15. Method for modifying the metabolite content of plants by modification of the transporter properties of the preferably inner mitochondrial membrane of a plant, a plant part, or a plant cell, comprising the insertion of a nucleic acid according to claim 1 or 2, or a fragment thereof, or a construct according to one of claims 5 to 7 containing the nucleic acid or the fragment thereof, into a plant cell and, potentially, the regeneration of a whole plant from this.

16. Method according to claim 15, whereby the transporter is an amino acid or carboxylic acid transporter.

17. Method for isolating at least one gene, encoding a plant or animal transporter of the inner mitochondrial membrane, from a gene bank, whereby an MCF-deficient yeast cell is transformed with nucleic acid sequences from a plant or animal gene bank, and a positive selection is performed.

18. Use of a nucleic acid according to claim 1 or 2, or a fragment thereof, for isolating homologous sequences from bacteria, fungi, plants, animals and/or humans.

19. Use of a nucleic acid according to claim 1 or 2 under the control of a regulatory element in antisense orientation or of a fragment thereof under the control of a regulatory element in antisense orientation for the inhibition of the expression of an endogenous MCF member in prokaryotic and/or eukaryotic cells.

20. Use of a nucleic acid according to claim 1 or 2, or a fragment thereof, for the production of transgenic crop plants with modified mitochondrial and/or plastid transport properties.

21. Use of a nucleic acid according to claim 1 or 2 for identifying inhibitors of the substance transport mediated by transporters of the inner mitochondrial membrane in mitochondria and/or plastids.
